SEQUENCE LISTING (89 sequences)

SEQ ID NO: 1 - protein, 6 aa, Artificial sequence
  Chemically synthesized
NCFVNN

SEQ ID NO: 2 - protein, 14 aa, Artificial sequence
  Chemically synthesized
AQNTVICSKLAAKC

SEQ ID NO: 3 - protein, 6 aa, Artificial sequence
  Chemically synthesized
KLGRRA

SEQ ID NO: 4 - protein, 22 aa, Artificial sequence
  Chemically synthesized
ESGLFKAKQCNGTSTCWCVNTA

SEQ ID NO: 5 - protein, 6 aa, Artificial sequence
  Chemically synthesized
CSERVR

SEQ ID NO: 6 - protein, 6 aa, Artificial sequence
  Chemically synthesized
LFHSKK

SEQ ID NO: 7 - protein, 10 aa, Artificial sequence
  Chemically synthesized
MAPPQVLAFG

SEQ ID NO: 8 - protein, 10 aa, Artificial sequence
  Chemically synthesized
QVLAFGLLLA

SEQ ID NO: 9 - protein, 14 aa, Artificial sequence
  Chemically synthesized
ITCSERVRTYWIII

SEQ ID NO: 10 - protein, 10 aa, Artificial sequence
  Chemically synthesized
TYWIIIELKH

SEQ ID NO: 11 - protein, 11 aa, Artificial sequence
  Chemically synthesized
IIELKHKAREK

SEQ ID NO: 12 - protein, 10 aa, Artificial sequence
  Chemically synthesized
SLRTALQKEI

SEQ ID NO: 13 - protein, 10 aa, Artificial sequence
  Chemically synthesized
ALQKEITTRY

SEQ ID NO: 14 - protein, 10 aa, Artificial sequence
  Chemically synthesized
DPKFITSILY

SEQ ID NO: 15 - protein, 10 aa, Artificial sequence
  Chemically synthesized
IADVAYYFEK

SEQ ID NO: 16 - protein, 10 aa, Artificial sequence
  Chemically synthesized
AYYFEKDVKG

SEQ ID NO: 17 - protein, 10 aa, Artificial sequence
  Chemically synthesized
DLDPGQTLIY

SEQ ID NO: 18 - protein, 10 aa, Artificial sequence
  Chemically synthesized
KAGVIAVIVV

SEQ ID NO: 19 - protein, 10 aa, Artificial sequence
  Chemically synthesized
IAVIVVVVMA

SEQ ID NO: 20 - protein, 10 aa, Artificial sequence
  Chemically synthesized
MAVVAGIVVL

SEQ ID NO: 21 - protein, 10 aa, Artificial sequence
  Chemically synthesized
AGIVVLVISR

SEQ ID NO: 22 - protein, 14 aa, Artificial sequence
  Chemically synthesized
MAPPQVLAFGLLLA

SEQ ID NO: 23 - protein, 14 aa, Artificial sequence
  Chemically synthesized
MNGSKLGRRAKPEG

SEQ ID NO: 24 - protein, 14 aa, Artificial sequence
  Chemically synthesized
WCVNTAGVRRTDKD

SEQ ID NO: 25 - protein, 10 aa, Artificial sequence
  Chemically synthesized
FGLLLAAATA

SEQ ID NO: 26 - protein, 10 aa, Artificial sequence
  Chemically synthesized
LAAATATFAA

SEQ ID NO: 27 - protein, 10 aa, Artificial sequence
  Chemically synthesized
TATFAAAQEE

SEQ ID NO: 28 - protein, 10 aa, Artificial sequence
  Chemically synthesized
AAAQEECVCE

SEQ ID NO: 29 - protein, 10 aa, Artificial sequence
  Chemically synthesized
EECVCENYKL

SEQ ID NO: 30 - protein, 10 aa, Artificial sequence
  Chemically synthesized
CENYKLAVNC

SEQ ID NO: 31 - protein, 10 aa, Artificial sequence
  Chemically synthesized
KLAVNCFVNN

SEQ ID NO: 32 - protein, 10 aa, Artificial sequence
  Chemically synthesized
NCFVNNNRQC

SEQ ID NO: 33 - protein, 10 aa, Artificial sequence
  Chemically synthesized
NNNRQCQCTS

SEQ ID NO: 34 - protein, 10 aa, Artificial sequence
  Chemically synthesized
QCQCTSVGAQ

SEQ ID NO: 35 - protein, 10 aa, Artificial sequence
  Chemically synthesized
TSVGAQNTVI

SEQ ID NO: 36 - protein, 10 aa, Artificial sequence
  Chemically synthesized
AQNTVICSKL

SEQ ID NO: 37 - protein, 10 aa, Artificial sequence
  Chemically synthesized
VICSKLAAKC

SEQ ID NO: 38 - protein, 10 aa, Artificial sequence
  Chemically synthesized
KLAAKCLVMK

SEQ ID NO: 39 - protein, 10 aa, Artificial sequence
  Chemically synthesized
KCLVMKAEMN

SEQ ID NO: 40 - protein, 10 aa, Artificial sequence
  Chemically synthesized
MKAEMNGSKL

SEQ ID NO: 41 - protein, 10 aa, Artificial sequence
  Chemically synthesized
MNGSKLGRRA

SEQ ID NO: 42 - protein, 10 aa, Artificial sequence
  Chemically synthesized
KLGRRAKPEG

SEQ ID NO: 43 - protein, 10 aa, Artificial sequence
  Chemically synthesized
RAKPEGALQN

SEQ ID NO: 44 - protein, 10 aa, Artificial sequence
  Chemically synthesized
EGALQNNDGL

SEQ ID NO: 45 - protein, 10 aa, Artificial sequence
  Chemically synthesized
QNNDGLYDPD

SEQ ID NO: 46 - protein, 10 aa, Artificial sequence
  Chemically synthesized
GLYDPDCDES

SEQ ID NO: 47 - protein, 10 aa, Artificial sequence
  Chemically synthesized
PDCDESGLFK

SEQ ID NO: 48 - protein, 10 aa, Artificial sequence
  Chemically synthesized
ESGLFKAKQC

SEQ ID NO: 49 - protein, 10 aa, Artificial sequence
  Chemically synthesized
FKAKQCNGTS

SEQ ID NO: 50 - protein, 10 aa, Artificial sequence
  Chemically synthesized
QCNGTSTCWC

SEQ ID NO: 51 - protein, 10 aa, Artificial sequence
  Chemically synthesized
TSTCWCVNTA

SEQ ID NO: 52 - protein, 10 aa, Artificial sequence
  Chemically synthesized
WCVNTAGVRR

SEQ ID NO: 53 - protein, 10 aa, Artificial sequence
  Chemically synthesized
TAGVRRTDKD

SEQ ID NO: 54 - protein, 10 aa, Artificial sequence
  Chemically synthesized
RRTDKDTEIT

SEQ ID NO: 55 - protein, 10 aa, Artificial sequence
  Chemically synthesized
KDTEITCSER

SEQ ID NO: 56 - protein, 10 aa, Artificial sequence
  Chemically synthesized
ITCSERVRTY

SEQ ID NO: 57 - protein, 10 aa, Artificial sequence
  Chemically synthesized
ERVRTYWIII

SEQ ID NO: 58 - protein, 10 aa, Artificial sequence
  Chemically synthesized
TYWIIIELKH

SEQ ID NO: 59 - protein, 10 aa, Artificial sequence
  Chemically synthesized
IIELKHKARE

SEQ ID NO: 60 - protein, 10 aa, Artificial sequence
  Chemically synthesized
KHKAREKPYD

SEQ ID NO: 61 - protein, 10 aa, Artificial sequence
  Chemically synthesized
REKPQDSKSL

SEQ ID NO: 62 - protein, 10 aa, Artificial sequence
  Chemically synthesized
YDSKSLRTAL

SEQ ID NO: 63 - protein, 10 aa, Artificial sequence
  Chemically synthesized
EITTRYQLDP

SEQ ID NO: 64 - protein, 10 aa, Artificial sequence
  Chemically synthesized
RYQLDPKFIT

SEQ ID NO: 65 - protein, 10 aa, Artificial sequence
  Chemically synthesized
ITSILYENNV

SEQ ID NO: 66 - protein, 10 aa, Artificial sequence
  Chemically synthesized
LYENNVITID

SEQ ID NO: 67 - protein, 10 aa, Artificial sequence
  Chemically synthesized
NVITIDLVQN

SEQ ID NO: 68 - protein, 10 aa, Artificial sequence
  Chemically synthesized
IDLVQNSSQK

SEQ ID NO: 69 - protein, 10 aa, Artificial sequence
  Chemically synthesized
QNSSQKTQND

SEQ ID NO: 70 - protein, 10 aa, Artificial sequence
  Chemically synthesized
QKTQNDVDIA

SEQ ID NO: 71 - protein, 10 aa, Artificial sequence
  Chemically synthesized
NDVDIADVAY

SEQ ID NO: 72 - protein, 10 aa, Artificial sequence
  Chemically synthesized
EKDVKGESLF

SEQ ID NO: 73 - protein, 10 aa, Artificial sequence
  Chemically synthesized
KGESLFHSKK

SEQ ID NO: 74 - protein, 10 aa, Artificial sequence
  Chemically synthesized
LFHSKKMDLT

SEQ ID NO: 75 - protein, 10 aa, Artificial sequence
  Chemically synthesized
KKMDLTVNGE

SEQ ID NO: 76 - protein, 10 aa, Artificial sequence
  Chemically synthesized
LTVNGEQLDL

SEQ ID NO: 77 - protein, 10 aa, Artificial sequence
  Chemically synthesized
GEQLDLDPGQ

SEQ ID NO: 78 - protein, 10 aa, Artificial sequence
  Chemically synthesized
GQTLIYYVDE

SEQ ID NO: 79 - protein, 10 aa, Artificial sequence
  Chemically synthesized
IYYVDEKAPE

SEQ ID NO: 80 - protein, 10 aa, Artificial sequence
  Chemically synthesized
DEKAPEFSMQ

SEQ ID NO: 81 - protein, 10 aa, Artificial sequence
  Chemically synthesized
PEFSMQGLKA

SEQ ID NO: 82 - protein, 10 aa, Artificial sequence
  Chemically synthesized
MQGLKAGVIA

SEQ ID NO: 83 - protein, 10 aa, Artificial sequence
  Chemically synthesized
VLVISRKKRM

SEQ ID NO: 84 - protein, 10 aa, Artificial sequence
  Chemically synthesized
SRKKRMAKYE

SEQ ID NO: 85 - protein, 10 aa, Artificial sequence
  Chemically synthesized
RMAKYEKAEI

SEQ ID NO: 86 - protein, 10 aa, Artificial sequence
  Chemically synthesized
YEKAEIKEMG

SEQ ID NO: 87 - protein, 10 aa, Artificial sequence sequence
  Chemically synthesized
EIKEMGEMHR

SEQ ID NO: 88 - protein, 10 aa, Artificial sequence
  Chemically synthesized
MGEMHRELNA

SEQ ID NO: 89 - protein, 314 aa, Homo sapiens [sequence omitted]

(19) United States
(12) Patent Application Publication
(10) Pub. No.: US 2007/0243201 A1
(43) Pub. Date: Oct. 18, 2007

(54) Method for Selecting Epitopes for Immunotherapy

(75) Inventors: Hans Loibner, Vienna (AT); Gottfried Himmler, Vienna (AT); Alois Jungbauer, Vienna (AT); Erich Wasserbauer, Vienna (AT); Manfred Schuster, Schrick (AT); Rainer Hahn, Vienna (AT); Astrid Durauer, Vienna (AT)

Correspondence Address: BIRCH STEWART KOLASCH & BIRCH, PO BOX 747, FALLS CHURCH, VA, 22040-0747, US

(73) Assignee: Igeneon Krebs-Immuntherapie Forschungs-und entwicklungs-AG, Wien (AT)

(21) Appl. No.: 10/559,131

(22) PCT Filed: Jun. 2, 2004
(86) PCT No.: PCT/AT04/00193

§ 371 Date: Dec. 1, 2005

(30) Foreign Application Priority Data
Jun. 2, 2003 (AT) A 852/2003

(51) Int. Cl. A61K 39/00; A61P 35/00; C07K 14/47; C07K 16/30; C07K 5/00; G01N 33/15

(52) U.S. Cl. 424/185.1; 436/501; 530/329

(57) ABSTRACT

The invention relates to a method for the selection of epitopes for immunotherapy, peptides obtained by said method, the use of said peptides as vaccines and diagnostics and an immune serum obtainable by said method.

The invention relates to a method for selecting epitopes for an immunotherapy, peptides obtainable by this method, and the use of the peptides as vaccines and as diagnostic agents, and an immune serum obtainable by this method.

Immunotherapy must not only be safe, i.e. the vaccines must not exhibit any toxicity, but it must also induce a protective immunity and generate as long-lived an immunological memory as possible. Particularly with cancer, one of the three most frequent causes of death in the industrialized countries, immunotherapy is an important prophylaxis and therapy approach.

Tumor-associated antigens (TAAs) often are the basis for the development of immunotherapeutical agents for the prophylaxis and/or treatment of cancer. TAAs are structures which preferably are expressed on the cell membrane of tumor cells, thereby enabling them to be distinguished from non-malignant tissue, and which therefore are seen as targets for the diagnostic and therapeutic applications of specific antibodies. Examples of tumor-associated carbohydrate structures are the Lewis antigens which are increasingly expressed in many types of epithelial cancer. Among them are Lewis x-, Lewis b- and Lewis y-structures as well as sialylated Lewis x-structures. Other carbohydrate antigens are GloboH structures, KH1, Tn antigen, TF antigen, the alpha-1,3-galactosyl epitope (Electrophoresis (1999), 20:362; Curr. Pharmaceutical Design (2000), 6:485, Neo-plasma (1996), 43:285).

Further TAAs are proteins which are particularly highly expressed on cancer cells, such as, e.g., CEA, TAG-72, MUC1, Folate Binding Protein A-33, CA125, EPCAM and PSA.

The direct therapeutical applications of antibodies against TAAs are based on passive immunotherapies, i.e. a specific antibody is systemically administered to cancer patients in a suitable amount and only has a therapeutic effect as long as its concentration within the organism is sufficiently high for this. The biological half-life of such agents depends on their structure and lasts from a few hours up to several days. Therefore, it is necessary to carry out repeated applications. However, when using xenogenic antibodies (e.g. murine monoclonal antibodies, MABs), this leads to undesired immune reactions which may neutralize a possible therapeutic effect and lead to dangerous side effects (anaphylactic reactions). Therefore, such immunotherapeutic agents can be administered for a limited time only.

Another approach for the immunotherapy of cancer is based on the selective activation of the immune system of cancer patients in order to fight malignant cells. This is attempted by the most varying forms of cancer vaccines. These include vaccinations with autologous or allogenic tumor cells, chemically or molecular-biologically modified autologous or allogenic tumor cells, isolated TAAs or TAAs prepared by chemical or molecular-biological methods, peptides derived therefrom, recently also vaccinations with DNA which codes for TAAs or for structures derived therefrom, etc. An alternative method is based on the use of anti-idiotypic antibodies for the vaccination against cancer. Suitable anti-idiotypic antibodies can immunologically mimic a TAA. Being foreign proteins (e.g. murine antibodies, goat-antibodies etc.), they induce a high immune response in humans after vaccination, in contrast to human tumor antigens proper which, being self-structures, often are only little immunogenic. Therefore, anti-idiotypic antibodies can be used as an immunogenic substitute of a tumor antigen for vaccination.

In contrast to the passive immunotherapy with anti-tumor antibodies, in principle very small amounts of a suitable vaccine suffice for the active specific immunotherapy of cancer so as to induce an immunity lasting for months up to years which, if it decreases, can be boostered again by booster vaccinations. Moreover, with an active immunization both a humoral and also a cellular immunity can be induced whose interaction can result in an effective protective activity.

In summary, the use of antibodies or of their derivatives hitherto employed in the immunotherapy of cancer substantially is based on two principles:
passive therapy with antibodies or their derivatives which are directed against TAAs. This involves the relatively specific destruction of tumor cells (Immunology Today (2000), 21:403-410; Curr. Opin. Immunol. (1997), 9:717);
active immunization (vaccination) with TAAs or antibodies or their derivatives which are directed against the idiotype of antibodies with a specificity against TAAs. The active vaccination triggers an immune response against TAAs. This immune response thus is also directed against the corresponding tumor cells (Ann. Med. (1999), 31:66; Immunobiol. (1999), 201:1).

In the course of the discovery and subsequent characterization of the most varying TAAs it has been found out that they have important functions for cancer cells. They allow the degenerated cells to carry out characteristic properties for the malignant phenotype, such as, e.g., an increased adhesion capacity, which are of great importance for the establishment of metastases. However, in certain stages, such antigens may very well be expressed on normal cells as well, where they are responsible for normal functions of these cells. Without a claim to completeness, a few examples of such antigens shall be listed here:
N-CAM (Neuronal Cell Adhesion Molecule) which often is expressed on tumors of neuronal origin and causes a homophilic adhesion (J. Cell Biol. 118 (1992), 937).
The Lewis Y carbohydrate antigen which appears on the plurality of the tumors of epithelial origin, but also has an important role during the fetal development of epithelial tissues. The expression of this antigen in lung cancer has been shown to be highly associated with an unfavorable prognosis, since Lewis Y positive cancer cells apparently have a higher metastatic potential (N. Engl. J. Med. 327 (1992), 14).
CEA (Carcino Embryonic Antigen) which frequently appears on epithelial tumors of the gastro-intestinal tract and has been identified as a self-adhesion molecule (Cell 57 (1989), 327).
EpCAM (Epithelial Cell Adhesion Molecule) which is expressed on nearly all the tumors of epithelial origin, yet also appears on many normal epitheliums, which has been characterized as a self-adhesion molecule and therefore can be classified as a pan-epithelial adhesion antigen (J. Cell Biol. 125 (1994), 437).

However, for designing an immunotherapy as efficiently and successfully as possible it is advantageous to isolate those epitopes which are particularly well suited for antibody recognition, since they are localized e.g. on the surface of a protein, or epitopes, which cause a particularly good antibody formation in immune sera. A simple and rapid selection method for such epitopes, or for isolated peptide sequences of these epitopes, respectively, would be advantageous for the success of immunotherapies.

In this respect, a combined approach has been described by Mintz et al., where peptides have been isolated from phage display random peptide libraries by means of immunoglobulins isolated from patients suffering from cancer of the prostate. These fingerprint methods have been used to isolate specific prognostic serological markers and their corresponding native antigens. A previous immunotherapy of these patients has not been described. The humoral immune system has been used for isolating a consensus sequence which can serve as a serological marker for survival chances (Nature Biotechn., 2003, 21, 57-63; Nature Biotechnol., 2003, 21, 37ff).

Mosolits et al. describe the binding of sera from patients suffering from colorectal carcinoma to immunogenic regions of the GA733-2 tumor associated antigen (TAA). A peptide sequence of 18 amino acids has been isolated as immunodominant B cell epitope, since 50% of the patients have antibodies against this peptide.

WO 97/15597 describes peptides whose amino acid sequence is derived from EpCAM and which bind to the MHC I molecule.

EP 0 326 423 describes vectors and methods for expressing the recombinant human adenocarcinoma antigen.

In these publications, the patients did not receive any immunotherapy, the immune serum was not derived from patients who had been treated with an effective vaccine.

Therefore, the present invention is based on the object to provide a method for selecting epitopes.

This object is achieved by providing the embodiments indicated in the claims.

According to the invention, a method for selecting epitopes for an immunotherapy is provided, wherein an immune serum is contacted with peptide sequences of an antigen having a length of at least 6 amino acids, and the binding of the immune serum to the peptide sequences is compared with the binding of a pre-serum.

The immune serum may be isolated from individuals who have been immunized with a vaccine. By the immunization, antibodies are developed against the antigen or against the antigenic structures with which immunization has been effected. Preferred antigens are selected from the group of the tumor associated antigens (TAAs). TAAs are structures which preferably are expressed on the cell membrane of tumor cells, thereby allow them to be distinguished from non-malignant tissue, and therefore are viewed as targets for the diagnostic and therapeutic applications of specific antibodies.

In an alternative embodiment, the TAAs are selected from the group of the self-adhesion molecules or cell adhesion molecules which can cause e.g. tumor cells to have a pronounced cell-cell adhesion. An example of a self-adhesion molecule is EPCAM.

Besides other physiological properties which distinguish them from normal cells, cancer cells practically always have an altered type of glycosylation (Glycoconj. J. (1997), 14:569; Adv. Cancer Res. (1989), 52-257; Cancer Res. (1996), 56:5309). Even though the alterations differ from tissue to tissue, it can be said that an aberrant glycosylation is typical of cancer cells.

Among the known tumor-associated carbohydrate structures, there are e.g. all the Lewis antigens which are increasingly expressed in many types of epithelial cancer. Besides Lewis-y structures, also Lewis-a and Lewis-b structures as well as sialylated Lewis-x structures belong thereto. Other carbohydrate antigens are Globo-H structures, KH1, Tn antigen and Sialyl Tn antigen, TF antigen and the alpha-1,3-galactosyl epitope (Electrophoresis (1999), 20:362; Curr. Pharmaceutical Design (2000), 6:485, Neoplasma (1996), 43:285).

Other TAAs are proteins which are particularly highly expressed by cancer cells, such as, e.g., CEA, TAG-72, MUC1, Folate Binding Protein A-33, CA125, Ep-CAM, HER-2/neu, PSA, MART, etc. (Sem. Cancer Biol. (1995), 6:321). Relevant TAAs often are surface antigens of epithelial cells which increasingly occur in growing cells, such as fetal tissue, and also tumor tissue. A special group of TAAs participate in the adhesion processes of the epithelial cells. Among the cellular adhesion proteins which are over-expressed on tumor cells are EpCAM, NCAM and CEA.

According to the present invention, the peptide sequences can be bound by known methods to a carrier and can be contacted with the immune serum, or the pre-serum, respectively. Preferably, the method according to the invention is carried out via epitope mapping by means of solid phase peptide synthesis and spot synthesis. For this purpose, the peptides are bound to the carrier material either directly or by means of spacer sequences.

The immune serum and the pre-serum can be purified via methods known from the prior art. For instance, purification may be carried out via protein G Sepharose, and the immunoglobulins thus obtained can be biotinylated or coupled with radioactive substances so as to simplify the detection method.

According to the invention, the pre-serum is a serum from individuals who have not received an immunotherapy with an antigen, with the peptides of which the serum is contacted later on.

According to the invention, immune serum is serum from individuals who have received an immunotherapy with an antigen, with the peptide sequences of which the serum subsequently is contacted. This may, e.g., be an immunization with epitopes against TAAs as have been mentioned above. Particularly preferably an immunotherapy was carried out with immunogenic antibodies, such as have been described e.g. in EP 1 140 168, EP 1 230 932, EP 0 644 947 and EP 0 528 767. A preferred antibody used for an active immunotherapy is an anti-EpCAM antibody, such as has been described in WO 00/41722 or in A599/2003.

According to the invention, an effective vaccine is understood to be a vaccine which is effective in the prophylaxis and/or in the therapy. With an active immunotherapy, the effectiveness preferably is proven in that the patient develops an immune response against the immunogenic substance. The measurement of the immune response can be effected by detecting the seroconversion in the patient's serum. The seroconversion is determined e.g. in that a differential measurement of the binding of the immunoglobulins from the patient's serum (pre-serum and immune serum) to the antigen used for immunizing is detected.

In a preferred embodiment, the treatment of patients with the effective vaccine leads to a lenghtening of the survival time and an increase of the survival rate, respectively, with cancer diseases, e.g. with colorectal or rectal cancer, by at least 10%, preferably at least 20%, preferably at least 30%, and particularly preferred at least 50%, as compared to patients without treatment with the effective vaccine.

The different binding patterns of pre- and immune sera to the peptides on the carrier material can be compared.

In a preferred embodiment, the method is carried out such that the binding of the immune serum to the peptide sequences is higher than the binding of the pre-serum. The different binding affinity may, e.g., be shown by an optically stronger signal which the immune serum shows in comparison with the pre-serum in the binding to the peptide sequence.

Alternatively, the stronger binding pattern may also be due to the fact that the amount of the antibody that binds to this peptide sequence is increased and leads to a stronger binding signal.

According to the invention, with the help of the claimed method also peptides can be isolated to which the immune serum binds differently than the pre-serum. Preferably, these are peptides from tumor-associated self-adhesion molecules, e.g. peptides of the EpCAM protein, preferably peptides from the extracellular domain of the EpCAM protein.

Particularly preferably, these are peptides with a length of at least 6 amino acids which are located within one of the following amino acid sequences, or are selected from one of the following amino acid sequences, respectively:

Asn Cys Phe Val Asn Asn (NCFVNN)
Ala Gln Asn Thr Val Ile Cys Ser Lys Ala Ala Lys
Cys (AQNTVICSKLAAKC)
Lys Leu Gly Arg Arg Ala (KLGRRA)
Glu Ser Gly Leu Phe Lys Ala Lys Gln Cys Asn Gly
Thr Ser Thr Cys Trp Cys Val Asn Thr Ala
(ESGLFKAKQCNGTSTCW-CVNTA)
Cys Ser Glu Arg Val Arg (CSERVR)
Leu Phe His Ser Lys Lys (LFHSKK)
Met Ala Pro Pro Gln Val Leu Ala Phe Gly
(MAPPQVLAFG)
Gln Val Leu Ala Phe Gly Leu Leu Leu Ala
(QVLAFGLLLA)
Ile Thr Cys Ser Glu Arg Val Arg Thr Tyr Trp Ile
Ile Ile (ITCSERVRTYWIII)
Thr Tyr Trp Ile Ile Ile Glu Leu Lys His
(TYWIIIELKH)
Ile Ile Glu Leu Lys His Lys Ala Arg Glu Lys
(IIELKHKA-REK)
Ser Leu Arg Thr Ala Leu Gln Lys Glu Ile
(SLRTALQKEI)
Ala Leu Gln Lys Glu Ile Thr Thr Arg Tyr
(ALQKEITTRY)
Asp Pro Lys Phe Ile Thr Ser Ile Leu Tyr
(DPKFITSILY)
Ile Ala Asp Val Ala Tyr Tyr Phe Glu Lys
(IADVAYYFEK)
Ala Tyr Tyr Phe Glu Lys Asp Val Lys Gly
(AYYFEKDVKG)
Asp Leu Asp Pro Gly Gln Thr Leu Ile Tyr
(DLDPGQTLIY)
Lys Ala Gly Val Ile Ala Val Ile Val Val
(KAGVIAVIVV)
Val Ile Ala Val Ile Val Val Val Val Met Ala
(IAVIVVVV-MA)
Met Ala Val Val Ala Gly Ile Val Val Leu
(MAVVAGIVVL)
Ala Gly Ile Val Val Leu Val Ile Ser Arg
(AGIVVLVISR).

Furthermore, these are also peptides having a length of at least 6 amino acids, selected from one of the following amino acid sequences of the EpCAM molecule, which may also be responsible for the self-adhesion:

Met Ala Pro Pro Gln Val Leu Ala Phe Gly Leu Leu
Leu Ala (MAPPQVLAFGLLLA)
Met Asn Gly Ser Lys Leu Gly Arg Arg Ala Lys Pro
Glu Gly (MNGSKLGRRAKPEG)
Trp Cys Val Asn Thr Ala Gly Val Arg Arg Thr Asp
Lys Asp (WCVNTAGVRRTDKD).

The length of the peptides will depend on their use. Preferably, their length will be at least 6 amino acids, and 30 amino acids at the most, preferably, 20 amino acids at the most, preferably 15 amino acids at the most, preferably 10 amino acids at the most.

According to the invention, the peptides can be bound to carrier molecules. Preferred carrier molecules are antibodies or antibody derivatives or antibody fragments, IgG2a antibodies or fragments thereof, KLH (Keyhole Limpet Haemocyanine), serum albumin etc. Particularly preferably, carrier molecules are used which have an immunogenic effect.

The term “immunogenic” defines any structure which will lead to an immune response in a specific host system. For instance, a murine antibody or its fragments may act highly immunogenic in the human organism, particularly if administering the former with adjuvants.

In a particular embodiment, a vaccine for the active immunotherapy can be prepared which contains the peptide together with a suitable adjuvant. For, it has proven successful to increase the immunogenicity of a vaccine by using adjuvants. For this vaccine adjuvants are suitable such as, e.g., aluminum hydroxide (Alu-Gel) or aluminum phosphate, growth factors, lymphokines, cytokines, such as IL-2, IL-12, GM-CSF, gamma-interferon, or complement factors, such as C3d, further liposome preparations or lipopolysaccharide from E. coli (LPS) or also formulations with additional antigens against which the immune system has already produced a strong immune response, such as tetanus toxoid, bacterial toxins, such as Pseudomonas exotoxins and derivatives of lipid A.

For the vaccine formulation, also further known methods for conjugation or denaturing of vaccine components may be used so as to further increase the immunogenicity of the active agent.

The vaccine containing an inventive peptide with a carrier molecule for the active immunization is preferably administered in an amount of between 0.01 μg and 10 mg. The immunogenicity of the vaccine can be further enhanced by xenogenic substances or derivatizing of the antibody which may serve as the carrier molecule for the peptide. The immunogenic dose of the vaccine preferably is between 0.01 μg and 750 μg, preferably between 100 μg and 1 mg most preferably 100 μg and 500 μg. A vaccine which is administered as a sustained release drug naturally will contain substantially higher amounts of immunogenic substance, e.g. at least 1 mg to 10 mg. In this case, the vaccine will be released in the body over an extended period of time.

Yet, according to the invention, the peptide can also be used as a target antigen for a passive immunotherapy. For this purpose, e.g. an antibody may be produced which contains this peptide as epitope and binds to EpCAM. In the passive immunotherapy, this antibody will be administered several times at intervals of from 1 to 2 weeks. The preferred amount of administered antibody will range between 1 mg and 1 g, preferably between 100 mg and 500 mg; preferably the administration will be an intravenous one.

Also an immune serum which comprises antibodies against epitopes which have been obtained according to the inventive method for the selection of epitopes is covered by the invention. Furthermore, also a vaccine against EpCAM expressing tumor cells which leads to the formation of an immune serum is covered by the invention. This vaccine may, e.g., contain peptides, antibodies, antibody derivatives or mimotopes, anti-idiotypic antibodies or plasmids that express the EPCAM protein, together with suitable carrier substances.

The peptides obtained according to the inventive method may also be used as antisense peptides. In case of cell-adhesion sequences, e.g. a peptide that binds to a region which is necessary for the cell adhesion may prevent such cell adhesion. In case of proteins, such as EpCAM or CEA, this may have the consequence that the cells can no longer combine to cell formations, and the formation of metastases is retarded, or prevented, respectively.

Also a diagnostic agent for detecting specific immunoglobulins containing a peptide obtained according to the inventive method, and a detection agent for determining the binding of an immune serum can be provided.

For instance, test strips to which peptides are coupled can be incubated with immune sera, and the immunoglobulin specificity can be detected by way of the coloration pattern. After incubation with biotinylated patient serum, the interaction with specific immunoglobulins is detected by a color reaction. This aims at indicating a built-up reactivity against specific protein regions in an immunization antigen, on the one hand, and at describing the specificity of a built-up immune response, on the other hand. Thus, e.g., a therapeutic effect can be correlated from the detected reactivity. As detection means, the peptides can be coupled with biotin groups or with radioactive markers, and a measurable signal can be read in that the radiation impinges on an X-ray film or on a radiation-sensitive film and causes a signal.

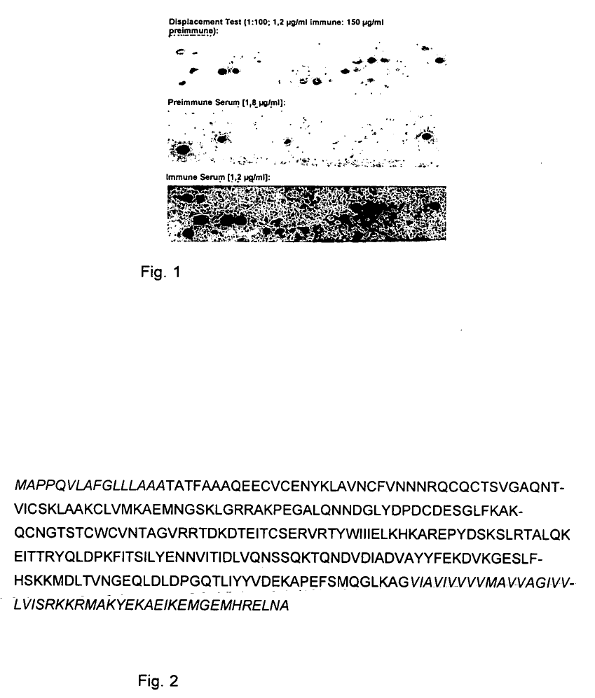

FIG. 1 by way of example shows a result of the Ep-CAM epitope mapping by means of solid phase synthesis.

FIG. 2 shows the amino acid sequence of the EpCAM molecule. The transmembrane region and the cytoplasmatic region are in italics.

### EXAMPLES

### Example 1

### Epitope Mapping by Means of a Solid Phase Peptide Synthesis

Purification of Sera

Protein G Sepharose Fast Flow (Amersham Biosciences) was packed into an HR 5 column (inner diameter 5 mm; Amersham Biosciences). The column was equilibrated with 5 column volumes of PBS buffer at a flow rate of 0.33 ml/min. Subsequently, serum was loaded at the same flow rate, followed by washing with further 5 column volumes. Elution was carried out with 0.2 M acetic acid +20% ethylene glycol, pH 2.7. The eluate was taken up in 1 M sodium bicarbonate buffer and thus brought to pH 8.6.

Biotinylation of the Sera and Antibodies, Respectively

One aliquot of the purified serum was incubated with NHS-LC biotin for 1 h at room temperature. Subsequently, the sample was re-buffered via a Sephadex G25 gel filtration column (PD-10, Amersham Biosciences) so as to separate the biotinylated protein from free protein, on the one hand, and to further transfer it into a medium suitable for the test system.

Spot Synthesis

The spot synthesis was carried out by means of a modified method according to Frank (Tetrahedron 48 (1992) 9217-9232). Whatman 540 cellulose was dried over night in the desiccator. The membrane was then functionalized for 3 h with 0.2 M Fmoc-β alanine, 0.24 M diisopropyl carbodiimide and 0.4 M 1-methyl-imidazole. After washing with 3× dimethylformamide (DMF), the Fmoc group was cleaved off by treatment with 20% piperidine in DMF. The peptides were synthesized by means of the AutoSpot Robot ASP222 (INTAVIS, Germany). As the first amino acid, always a further β-alanine spacer was introduced. After the first β-alanine coupling, the remaining free amino groups were acetylated with 2% acetic anhydride. As the amino acids, Fmoc-protected Opfp esters of the amino acids (O-pentafluorophenyl, Novabiochem), dissolved in N-methyl pyrrolidone at a concentration of 0.3 M were used. Each amino acid was spotted twice per cycle. After each cycle, the Fmoc group was cleaved off with piperidine, as described above, and then the membrane was washed 6× with DMF. After the synthesis of the last amino acid, it was again acetylated, and then the side protection groups were cleaved off. At first, the membrane was treated with 90% trifluoroacetic acid, 3% triisobutyl silane, 1% phenol, 2% water and 4% dichloromethane for 30 minutes. Subsequently, the membrane was washed 5× with DCM, 3× with DMF and methanol and dried. This was followed by a further cleavage by treatment with 50% trifluoroacetic acid, 3% triisobutyl silane, 1% phenol, 2% water and 44% dichloromethane for two hours. Subsequently, it was washed again with DCM, DMF and methanol, and the membrane was stored at −20° C.

In order to cover the amino acid sequence of the extra-cellular part of EpCAM, 77 decapeptides with 6-fold overlaps each were immobilized on cellulose membranes via double-β-alanine linkers. The N-terminal side groups were protected by means of Fmoc strategy.

TABLE 1

Amino acid sequence of the decapeptides syn-
thesized for the epitope mapping of Ep-CAM

| | |
|---|---|
| # | AA Sequence |

| | |
|---|---|
| 1 | MAPPQVLAFG |
| 2 | QVLAFGLLLA |
| 3 | FGLLLAAATA |
| 4 | LAAATATFAA |
| 5 | TATFAAAQEE |
| 6 | AAAQEECVCE |
| 7 | EECVCENYKL |
| 8 | CENYKLAVNC |
| 9 | KLAVNCFVNN |
| 10 | NCFVNNNRQC |
| 11 | NNNRQCQCTS |
| 12 | QCQCTSVGAQ |
| 13 | TSVGAQNTVI |
| 14 | AQNTVICSKL |
| 15 | VICSKLAAKC |
| 16 | KLAAKCLVMK |
| 17 | KCLVMKAEMN |
| 18 | MKAEMNGSKL |
| 19 | MNGSKLGRRA |
| 20 | KLGRRAKPEG |
| 21 | RAKPEGALQN |
| 22 | EGALQNNDGL |
| 23 | QNNDGLYDPD |
| 24 | GLYDPDCDES |
| 25 | PDCDESGLFK |
| 26 | ESGLFKAKQC |
| 27 | FKAKQCNGTS |
| 28 | QCNGTSTCWC |
| 29 | TSTCWCVNTA |
| 30 | WCVNTAGVRR |
| 31 | TAGVRRTDKD |
| 32 | RRTDKDTEIT |
| 33 | KDTEITCSER |
| 34 | ITCSERVRTY |
| 35 | ERVRTYWIII |
| 36 | TYWIIIELKH |
| 37 | IIELKHKARE |
| 38 | KHKAREKPYD |
| 39 | REKPQDSKSL |
| 40 | YDSKSLRTAL |
| 41 | SLRTALQKEI |
| 42 | ALQKEITTRY |
| 43 | EITTRYQLDP |
| 44 | RYQLDPKFIT |
| 45 | DPKFITSILY |
| 46 | ITSILYENNV |
| 47 | LYENNVITID |
| 48 | NVITIDLVQN |
| 49 | IDLVQNSSQK |
| 50 | QNSSQKTQND |
| 51 | QKTQNDVDIA |
| 52 | NDVDIADVAY |
| 53 | IADVAYYFEK |
| 54 | AYYFEKDVKG |
| 55 | EKDVKGESLF |
| 56 | KGESLFHSKK |
| 57 | LFHSKKMDLT |
| 58 | KKMDLTVNGE |
| 59 | LTVNGEQLDL |
| 60 | GEQLDLDPGQ |
| 61 | DLDPGQTLIY |
| 62 | GQTLIYYVDE |
| 63 | IYYVDEKAPE |
| 64 | DEKAPEFSMQ |
| 65 | PEFSMQGLKA |
| 66 | MQGLKAGVIA |
| 67 | KAGVIAVIVV |
| 68 | IAVIVVVVMA |
| 69 | VVVVMAVVAG |
| 70 | MAVVAGIVVL |
| 71 | AGIVVLVISR |
| 72 | VLVISRKKRM |
| 73 | SRKKRMAKYE |
| 74 | RMAKYEKAEI |
| 75 | YEKAEIKEMG |
| 76 | EIKEMGEMHR |
| 77 | MGEMHRELNA |

Since the major immune response is provided in the form of IgG, IgG was purified from the sera via protein G Sepharose. After acidic elution with 0.2 M acetic acid in the presence of 20% ethylene glycol, the indvidual fractions were taken up in 1 M sodium carbonate so as to increase the pH as rapidly as possible. These conditions also allow for the directly following biotinylation of the purified IgG without previous re-buffering. After a dition of the biotinylating reagent (biotine-amidocaproate N-hydroxysuccinimide ester, NHS-LC-Biotin, ECL protein biotinylation module, RPN 2202, Amersham Biosciences) in a 40-fold molar excess to the IgG-solution and a 1 hour incubation at room temperature, the excess of biotin was removed via small Sephadex G 25 columns, and at the same time the biotinylated material was re-buffered in PBS. The concentration of the biotinylated IgG was determined via photometric absorption measurement at 280 nm, and after the addition of 0.2% sodium azide the material was stored at 4° C.

Development of the Membranes:

Carrying out the membrane development is very similar to a Western blot, and it is made up of the same steps: the cellulose membranes are conditioned in 20% methanol and blocked with 3% BSA/PBS-T. This was followed by the incubation with the biotinylated sera. After a washing step, there followed an incubation with Streptavidin-HRP conjugate (ECL protein biotinylation module, RPN 2202, Amersham Biosciences), so as to then induce the detectable light development via oxidation of the peroxidase in the presence of an enhancer solution. The membranes were measured immediately after development and at constant measurement duration. The cocurrent blank value membranes were only incubated with the dilution buffer instead of biotinylated IgG and subsequently incubated with Streptavidin-HRP.

In summary, after having optimized the blank membranes and the regenerating ability, there resulted the following conditions for the following tests:

TABLE 2
Optimized conditions for epitope mapping

| Step | Solution | Duration |
|---|---|---|
| Conditioning | 20% Methanol | 2 h |
| Blocking | 3% BSA/PBS with 0.1% | over night, 4° C. |
| | Tween 20 (PBS-T) | |
| Washing I | PBS-T | rinsing |
| Biotinylated | 1% BSA/PBS-T | 1 h |
| sera | [1-3 μg/ml] | |
| Washing II | PBS-T | rinsing, 3 × 5′, |
| | | 1 × 15′, 1 × 30′ |
| Streptavidin- | PBS-T with 0.8M NaCl | 1 h |
| HRP | final concentration | |
| | [0.25 μg/ml] | |
| Washing III | PBS-T with 0.8M NaCl | rinsing, 3 × 5′, |
| | final concentration | 1 × 15′, 1 × 30′ |
| Substrate | Peroxide - | 5′ |
| addition | Enhancer 1:1 | |
| Measuring | — | 30″, 1′, 5′, 10′ |

In order to increase the informative value and the comparability of the method, a displacement test was carried out in addition to the analysis of the biotinylated preimmune and immune sera. For this purpose, one part of the purified preimmune sera each was not biotinylated, but only re-buffered in PBS.

Furthermore, in each case the analyses of all the sera of an individual as well as the displacement test were carried out on consecutive days on one and the same membrane. For checking the successful regeneration of the membranes, blank membranes were routinely developed between the individual tests.

The results can be taken from FIG. 1. The immune serum was from a patient who had been subjected to an active immunotherapy with mAb17-1A, an EpCAM antibody. The immune serum was taken 71 days after immunization. The pre-serum was taken on day 1, i.e. shortly before the treatment was started. As has been shown, immune serum increasingly binds to the 10 mer peptides 1-2, 34-36, 42, 45 53, 61, 68-71, whereas the pre-serum does not show this binding or shows it to a slight degree only.

An increase in the salt concentration to 0.8 M NaCl final concentration in the dilution buffer of the conjugate with a simultaneous reduction of the conjugate concentration to 0.25 μg/ml allowed for an optimum reduction of the detectable spot on the blank membranes with a simultaneous detectability of the bound serum IgGs.

Therefore, as the optimized conditions for the detection step of the method using Streptavidin-HRP conjugate, the following was chosen:
Reagent: 0.25 pg/ml Streptavidin-HRP, diluted 1:6400 (Amersham Biosciences)
1% BSA/PBS-T with 0.8 M NaCl final concentration Duration of incubation: 1 h.

1. A method for selecting epitopes for an immunotherapy, characterized in that an immune serum A each and a pre-serum are contacted with peptide sequences of an antigen having a length of at least 6 amino acids, and the binding of the immune serum to the peptide sequences is compared with the binding of the pre-serum to the peptide sequences, wherein the epitopes are selected on the basis of different binding patterns.

2. A method according to claim 1, characterized in that the binding of the immune serum to the peptide sequences is stronger than the binding of the pre-serum.

3. A method according to claim 1 or 2, characterized in that the immune serum is derived from patients who have been treated with a vaccine effective against said antigen.

4. An isolated peptide having a length of at least 6 amino acids, obtainable by a method according to claim 1.

5. An isolated peptide according to claim 4, characterized in that it binds to the extra-cellular domain of a self-adhesion protein.

6. A peptide according to claim 5, characterized in that it binds to the extra-cellular domain of an EpCAM molecule.

7. A peptide according to claim 4, characterized in that it is located within an amino acid sequence selected from the group consisting of
Asn Cys Phe Val Asn Asn (NCFVNN)
Ala Gln Asn Thr Val Ile Cys Ser Lys Ala Ala Lys
Cys (AQNTVICSKLAAKC)
Lys Leu Gly Arg Arg Ala (KLGRRA)
Glu Ser Gly Leu Phe Lys Ala Lys Gln Cys Asn Gly
Thr Ser Thr Cys Trp Cys Val Asn Thr Ala
(ESGLFKAKQCNGTSTCWCVNTA)
Cys Ser Glu Arg Val Arg (CSERVR)
Leu Phe His Ser Lys Lys (LFHSKK)
Ile Thr Cys Ser Glu Arg Val Arg Thr Tyr Trp Ile
Ile Ile (ITCSERVRTYWIII)
Thr Tyr Trp Ile Ile Ile Glu Leu Lys His
(TYWIIIELKH)
Ile Ile Glu Leu Lys His Lys Ala Arg Glu Lys
(IIELKHKAREK)
Ser Leu Arg Thr Ala Leu Gln Lys Glu Ile
(SLRTALQKEI)
Ala Leu Gln Lys Glu Ile Thr Thr Arg Tyr
(ALQKEITTRY)
Ile Ala Asp Val Ala Tyr Tyr Phe Glu Lys
(IADVAYYFEK)
Ala Tyr Tyr Phe Glu Lys Asp Val Lys Gly
(AYYFEKDVKG)
Asp Leu Asp Pro Gly Gln Thr Leu Ile Tyr
(DLDPGQTLIY)
Val Ile Ala Val Ile Val Val Val Val Met Ala
(IAVIVVVVMA)
Met Ala Val Val Ala Gly Ile Val Val Leu
(MAVVAGIVVL)
Ala Gly Ile Val Val Leu Val Ile Ser Arg
(AGIVVLVISR)
Met Ala Pro Pro Gln Val Leu Ala Phe Gly Leu Leu
Leu Ala (MAPPQVLAFGLLLA)
Met Asn Gly Ser Lys Leu Gly Arg Arg Ala Lys Pro
Glu Gly (MNGSKLGRRAKPEG)
Trp Cys Val Asn Thr Ala Gly Val Arg Arg Thr Asp
Lys Asp (WCVNTAGVRRTDKD).

8. A peptide according to claim 4, characterized in that it is coupled to a carrier molecule.

9. A peptide according to claim 8, characterized in that the carrier molecule is an antibody, an antibody derivative, KLH or serum albumin.

10. A peptide according to claim 8, characterized in that the carrier molecule is immunogenic.

11. A vaccine, comprising a peptide according to claim 4 and a suitable adjuvant.

12. The use of a peptide according to claim 4 as target antigen for a passive immunotherapy

13. The use of a peptide according to claim 4 for producing an antibody for recognizing a protein containing the peptide.

14. A diagnostic agent comprising a peptide according to claim 4 and a detection means for determining the binding of an immune serum.

15. The use of a method according to claim 1 for detection cell-specific different expression patterns of a protein.

16. The use of a method according to claim 1 for detecting different glycosylation patterns of a protein.

17. The use of a peptide according to claim 4, characterized in that the peptide is used as antisense peptide.

18. An immune serum comprising antibodies against epitopes that have been selected according to a method according to claim 1.

19. A vaccine against EpCAM expressing tumor cells, which leads to the formation of an immune serum as defined in claim 1.
